(19) United States
(12) Patent Application Publication
(10) Pub. No.: US 2026/0069609 A1
(43) Pub. Date: Mar. 12, 2026

(54) ULTRAVIOLET DEVICE

(71) Applicant: ESSE MAURITIUS, Cascavelle (MU)

(72) Inventors: Trevor STEYN, Cascavelle (MU); Kyle Timothy GROENEWALD, Cascavelle (MU); Thomas John THERON, Cascavelle (MU)

(21) Appl. No.: 18/826,566

(22) Filed: Sep. 6, 2024

(51) Int. Cl. A61K 31/575; A61K 9/00

(52) U.S. Cl. CPC A61K 31/575; A61K 9/0014

(57) ABSTRACT

An ultraviolet light emitting device for irradiating a topical product. The device may include a housing, a recess defined by the housing. The recess may be configured to receive the topical product. An ultraviolet light source may be capable of irradiating the topical product within an irradiation zone of the recess. The light source and irradiation zone are substantially opposite one another so as to enable irradiation of the topical product by the ultraviolet light source to operatively convert a sterol in the topical product to an intermediate of vitamin D.

### TECHNICAL FIELD

This disclosure relates to skincare products, devices and methods. More particularly, but not exclusively, the disclosure relates to a device and method that facilitates the administration of previtamin D to human skin.

### BACKGROUND

Vitamin D is a secosteroid with many biological effects that are essential for human health. Low systemic levels of vitamin D has been shown to cause bone deformities such as rickets in children and osteoporosis or osteomalacia in adults.

Low systemic vitamin D levels, as determined by the concentration of 25-hydroxycholecalciferol in the blood, are associated with an increased risk of cardiovascular disease, hypertension, diabetes, insulin resistance, autoimmune diseases such as multiple sclerosis, various forms of cancer, respiratory infections, depression, and cognitive impairment.

The normal human diet delivers very little vitamin D, typically delivering between 0 and 200 international units (I.U.) per day. Exposure to direct sunlight, which includes ultraviolet (UV) light particularly UVB light, is required for vitamin D synthesis in human skin and is the primary route through which vitamin D is synthesised in the human body. Specially, vitamin D3 is produced in the skin from the precursor, 7-dehydrocholesterol, when exposed to UV irradiation which breaks the B ring to form previtamin D ((3S,6Z)-9,10-secocholesta-5(10),6,8-trien-3-ol). Previtamin D isomerizes to vitamin D3 at body temperature over time. Additionally, previtamin D can also isomerize to tachysterol and lumisterol, neither of which present toxicity risks and serve as part of an evolved protection mechanism to prevent vitamin D toxicity in the human body when there is high UV exposure.

However, previtamin D3 is not stable at room temperature and therefore cannot be incorporated into products with a commercially viable shelf life.

Vitamin D may also be obtained from oral supplements of vitamin D2 or D3. However, with the exception of rickets, oral supplementation with vitamin D has been shown to be ineffective in reducing the risk of many of the diseases associated with low vitamin D.

Although required for vitamin D production, exposure to UV light (sunlight or artificial UV light) is associated with an increased rate of ageing, DNA damage, and the development of melanoma or skin cancer. Due to these associated risks, the human population has been shown to increasingly limit their exposure to sunlight and UV light.

The average person in an industrialised economy now spends 93% of their time indoors or inside a vehicle. Window glass from buildings or vehicles largely blocks UVB light preventing the production of meaningful levels of vitamin D indoors. Furthermore, many cultures value lighter skin tones, and some cultures require that the entire or part of the human body be covered year-round, both of which in turn further contribute to low sun exposure. This reduction in sunlight or UV light exposure, whether to avoid the associated risks of UV exposure or due to lifestyle changes in the past 200 years, has led to widespread vitamin D deficiency across the human population. It is estimated that more than 1 billion people globally suffer from vitamin D deficiency, and more than 50% of the world's population has insufficient vitamin D levels.

Synthetic vitamin D has been employed as an effective rodenticide, and oral supplementation with synthetic vitamin D carries certain risks. Over-supplementation with synthetic vitamin D causes hypercalcaemia, and there are concerns that even moderate doses may increase the risk of plaque buildup in arteries.

Direct application of topical products containing vitamin D3 can effectively raise systemic levels, but these products are often classified scheduled drugs in many countries due to the risk of toxicity after overapplication. Furthermore, these products share the same risks and efficacy profile as the oral supplements mentioned above.

Artificial UV light irradiated onto human skin has been used to supplement vitamin D levels since the early 1900s. These devices, which originally utilised specially designed fluorescent bulbs, have now been replaced by LED variants. However, as mentioned, UV light poses various risks to human health and may also cause burn wounds or eye damage if used incorrectly.

Given the risks associated with UV light exposure and the limitations of artificial vitamin D supplementation, there is a critical need for a solution that facilitates the natural synthesis of vitamin D without the associated toxicity risks. As the global population continues to shift towards an indoor-oriented, industrialized lifestyle, the demand for a simple and low-risk solution for vitamin D synthesis has never been more urgent.

The preceding discussion of the background is intended only to facilitate an understanding of the present disclosure. It should be appreciated that the discussion is not an acknowledgment or admission that any of the material referred to was part of the common general knowledge in the art as at the priority date of the application.

### SUMMARY

In accordance with an aspect of the disclosure there is provided an ultraviolet light emitting device for irradiating a topical product, the device comprising a housing, a recess defined by the housing and configured to receive the topical product, and an ultraviolet light source capable of irradiating the topical product within an irradiation zone of the recess, wherein the light source and irradiation zone are substantially opposite one another so as to enable irradiation of the topical product by the ultraviolet light source to operatively convert a sterol in the topical product to an intermediate of vitamin D.

The ultraviolet light source may alternatively, or in addition enable irradiation of the topical product to operatively convert a precursor 5,7-dehydrosterol in the topical product to an intermediate of vitamin D. The intermediate of vitamin D may be previtamin D.

The housing may comprise at least a first housing body and a second housing body. The first and second housing bodies may be operable between an open or disengaged condition and a closed or engaged condition.

The sterol may be a 5,7-dehydrosterol or 7-dehydrocholesterol. The intermediate of vitamin D may be an intermediate isomer or previtamin D or tachysterol or lumisterol.

The ultraviolet light source may be capable of emitting ultraviolet B (UVB) light. The UVB light may have a wavelength in the range of 280 nanometres up to 315 nanometres, or 285 up to 310 nanometres. The ultraviolet light source may be a light emitting diode (LED) light.

The light source may be located at the first housing body, and the irradiation zone at the second housing body. The light source and irradiation zone may both be located at one of the first and second housing bodies.

The housing may include an engagement structure configured to releasably secure the first and second body. The engagement structure may be in the form of one or more magnet(s) provided in either of the first and second housing bodies. The one or more magnet(s) may be arranged to releasably engage magnetic material in the other of the first and second housing bodies, and/or wherein the one or more magnet(s) may be arranged to releasably engage a corresponding magnet in the other of the first and second housing bodies.

The engagement structure may be an array of magnets disposed along a periphery of an internal surface of either of the bodies and configured to magnetically secure the bodies to one another.

The engagement structure may facilitate pivotal movement and/or sliding movement of the first and second housing between the open or disengaged condition and the closed or engaged condition.

The device may include a safety device or a safety mechanism to prevent accidental irradiation. The safety device may comprise a magnetically activated switch, configured to deactivate the ultraviolet light source when the first and second bodies are disengaged. The switch may comprise a hall effect sensor and semiconductor, or other magnetic switch.

The device may include a power source. The power source may be one or more of a battery, a rechargeable battery, a Universal Serial Bus (USB) port for connection to an external power source, and a mains power source.

The conversion rate of 7-dehydrocholesterol in a fatty acid ester solvent to an intermediate of Vitamin D may occur at a rate of about 10% to 99% in 2 seconds to 24 hours, or it may occur substantially in 10 seconds up to 10 minutes.

In accordance with another exemplary aspect of the present disclosure there is provided a method for irradiating a topical product, the method comprising: providing an ultraviolet light emitting device as defined above; applying topical product into the recess of the housing; and engaging the first and second housing bodies with one another so as to cause irradiation of the topical product.

The irradiation of the topical product may operatively convert a sterol in the topical product to previtamin D.

The step of engaging the first and second housing bodies may be facilitated by pivotal movement and/or sliding movement of an engagement structure between the open or disengaged condition and the closed or engaged condition.

The step of engaging the first and second housing bodies may include activation of a safety device including a magnetically activated switch to prevent accidental irradiation, wherein irradiation is deactivated when the first and second bodies are disengaged.

In accordance with another exemplary aspect of the present disclosure there is provided a topical product for use with the device above, comprising 7-dehydrocholesterol dissolved in a skin-compatible solvent.

The skin-compatible solvent may have low ultraviolet absorption in the range 285 to 310 nanometres. The skin-compatible solvent may be a fatty acid ester, an alcohol, or an ester of isoamyl alcohol and a fatty acid.

The fatty acid may be lauric acid. The solvent may be one of isoamyl laurate, isopropyl palmitate, isopropyl myristate, octyl dodecanol, C12-C15 alkyl benzoate, or combinations thereof.

Embodiments of the technology will now be described, by way of example only, with reference to the accompanying drawings.

### BRIEF DESCRIPTION OF THE DRAWINGS

In the drawings:

FIG. 1 is three-dimensional view of an example embodiment of an ultraviolet device;

FIG. 2 is an exploded view of the ultraviolet device of FIG. 1;

FIG. 3 is a bottom view of a top cover of the first housing body of the ultraviolet device of FIG. 1;

FIG. 4 is a section view from the side of a top cover of the first housing body of the ultraviolet device of FIG. 1;

FIG. 5 is a three-dimensional view of the ultraviolet device of FIG. 1 with the top cover of the first housing body removed;

FIG. 6 is a three-dimensional view of an irradiation structure and base of the first housing body of the ultraviolet device of FIG. 1;

FIG. 7 is a top view of the irradiation structure of the ultraviolet device of FIG. 1;

FIG. 8 is semi-transparent a top view of an exemplary embodiment of a printed circuit (PC) board included in the irradiation structure of the ultraviolet device of FIG. 1 with the power source and control button indicated by broken lines;

FIG. 9 is a side view of the irradiation structure of the ultraviolet device of FIG. 1;

FIG. 10 is a diagrammatic semi-transparent three-dimensional view of the exemplary embodiment of a printed circuit (PC) board included in the irradiation structure of the ultraviolet device of FIG. 1 with the power source and control button indicated by broken lines;

FIG. 11 is a bottom view of the base of the first housing body of the ultraviolet device of FIG. 1;

FIG. 12 is a section view from the side of the base of the first housing body of the ultraviolet device of FIG. 1;

FIG. 13 is a top view of the second housing body of the ultraviolet device of FIG. 1;

FIG. 14 is a section view from the side of the second housing body of the ultraviolet device of FIG. 1;

FIG. 15 is a sectional view from the front of the ultraviolet device of FIG. 1 illustrating irradiation of a topical product at an irradiation zone;

FIG. 16 is a sectional view from the side of the ultraviolet device of FIG. 1 illustrating irradiation of a topical product at an irradiation zone; and

FIG. 17 is an exemplary flow diagram illustrating a method for irradiating a topical product.

### DETAILED DESCRIPTION WITH REFERENCE TO THE DRAWINGS

The present disclosure relates to the field of skincare products, devices and methods. More particularly, a device and method that facilitates the administration of previtamin D to human skin and a topical product for use with the device and method. The present disclosure extends to the conversion of a sterol, such as 7-Dehydrocholesterol or provitamin D, to an intermediate of vitamin D, such as previtamin D, which converts to vitamin D at body temperature over time following application of the irradiated topical product, including the previtamin D, onto the skin. An ultraviolet light emitting device for irradiating a topical product is provided, the device comprising a housing, a recess defined by the housing and configured to receive the topical product, and an ultraviolet light source capable of irradiating the topical product within an irradiation zone of the recess.

The light source and irradiation zone are located substantially opposite one another within the housing so as to enable irradiation of the topical product by the ultraviolet light source to operatively convert a sterol in the topical product to previtamin D.

Alternatively, or in addition, the ultraviolet light source may enable irradiation of the topical product to operatively convert a 5,7-dehydrosterol in the topical product to an intermediate of vitamin D, such as previtamin D.

The housing may comprise at least a first housing body and a second housing body. The first and second housing bodies may be operable between an open or disengaged condition and a closed or engaged condition.

The housing may include an engagement structure configured to releasably secure the first and second body and facilitate movement of the bodies between the open or disengaged condition and a closed or engaged condition. This movement may be a pivotal movement or a sliding movement.

The engagement structure may be in the form of one or more magnet(s) provided in either of the first and second housing bodies. The one or more magnet(s) may be arranged to releasably engage magnetic material in the other of the first and second housing bodies, and/or wherein the one or more magnet(s) may be arranged to releasably engage a corresponding magnet in the other of the first and second housing bodies.

The engagement structure may be an array of magnets disposed along a periphery of an internal surface of either of the bodies and configured to magnetically secure the bodies to one another.

The ultraviolet light source may be a light emitting diode (LED) light. The light source may be located at the first housing body, and the irradiation zone at the second housing body. The light source and irradiation zone may both be located at one of the first and second housing bodies.

The device may include a safety device or a safety mechanism configured to protect a user from accidental ultraviolet irradiation by deactivating the ultraviolet light source when the ultraviolet light emitting device is in the disengaged condition. The safety device may also include alerting a user when the ultraviolet light emitting device is in the engaged condition and irradiation is occurring. The safety device may comprise a safety or magnetically activated switch which may deactivate the ultraviolet light source when the first and second bodies are disengaged so as to prevent ultraviolet light from escaping the housing once irradiation is activated or when the first and second housing bodies are in the engaged condition. The switch may comprise a hall effect sensor and semiconductor, reed switch, or other magnetic switch. It is envisaged that in alternative embodiments, a button switch may be used as alternative to the magnetically activated switch to activate and deactivate the ultraviolet light source irrespective of if the bodies are in the engaged or disengaged condition.

The device may include a power source. The power source may be one or more of a battery, a rechargeable battery, a Universal Serial Bus (USB) port for connection to an external power source, and a mains power source.

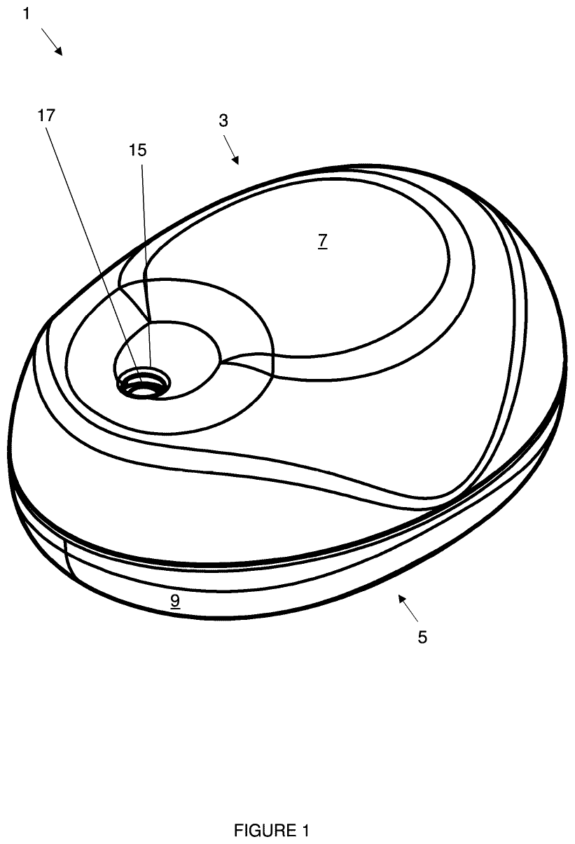

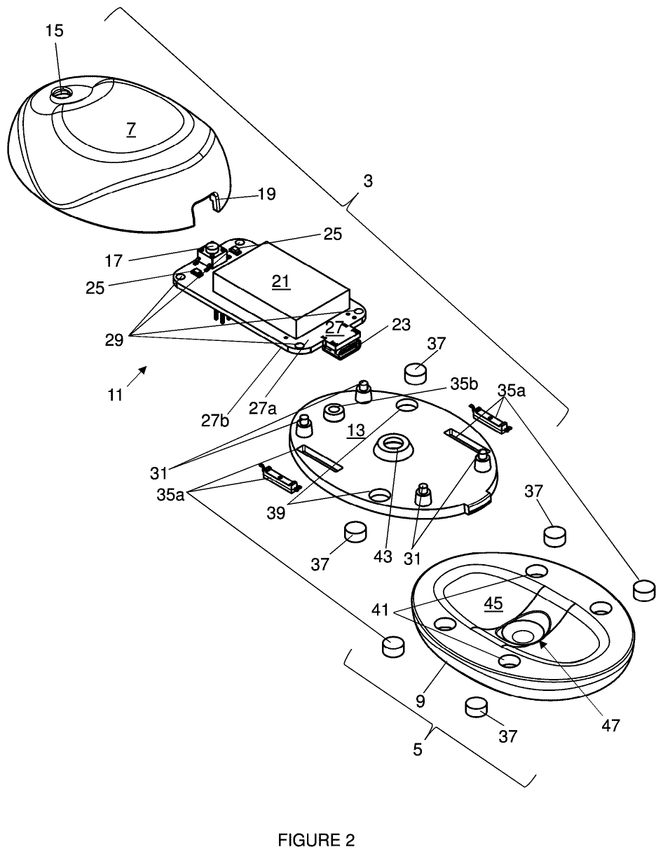

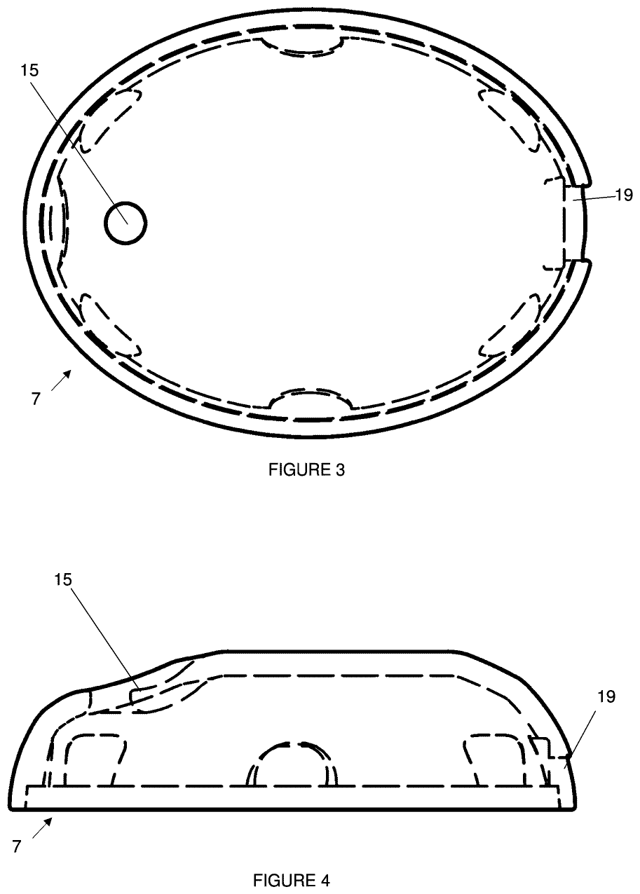

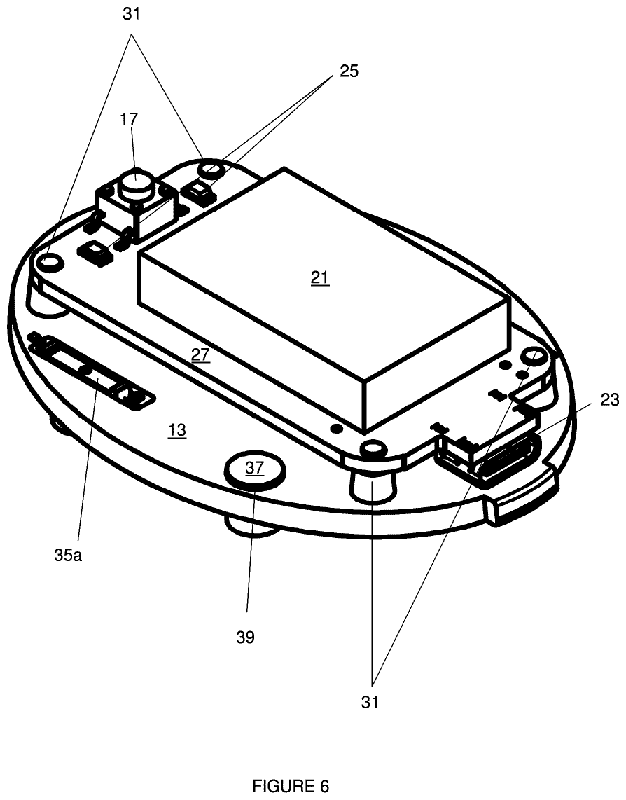

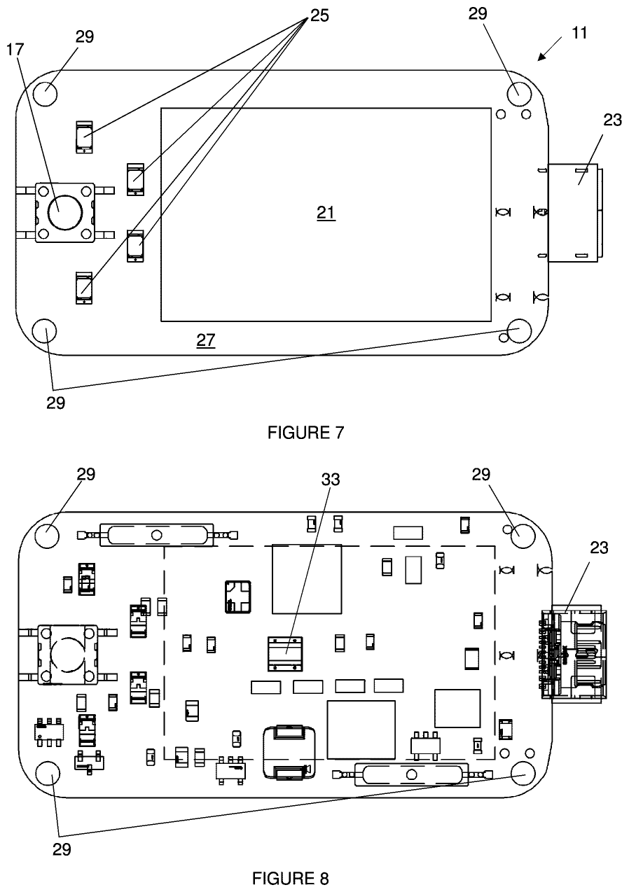

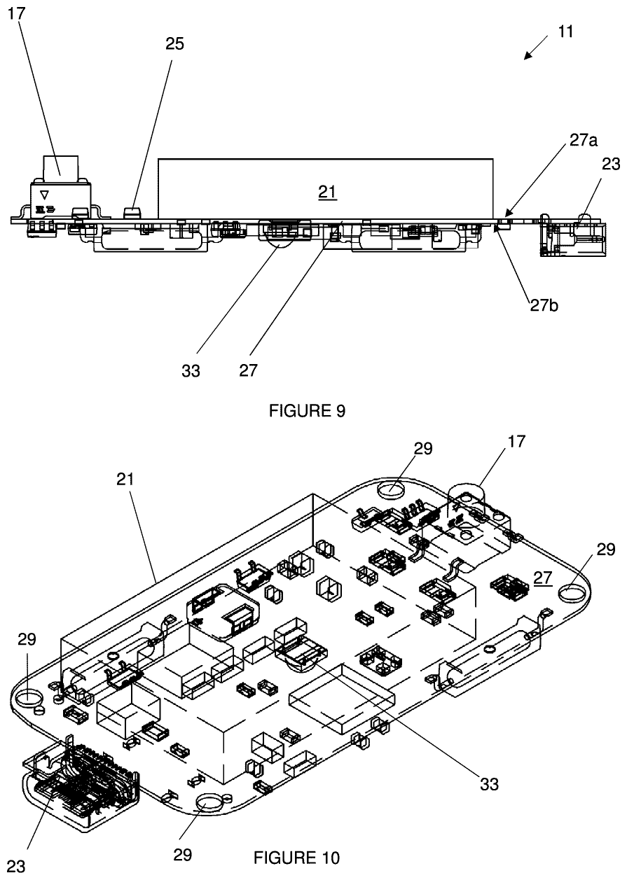

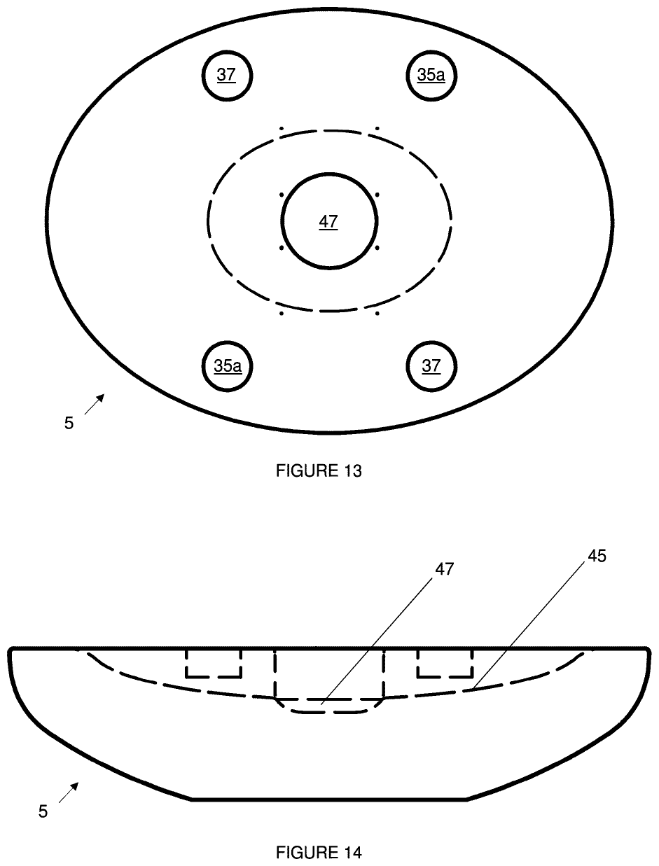

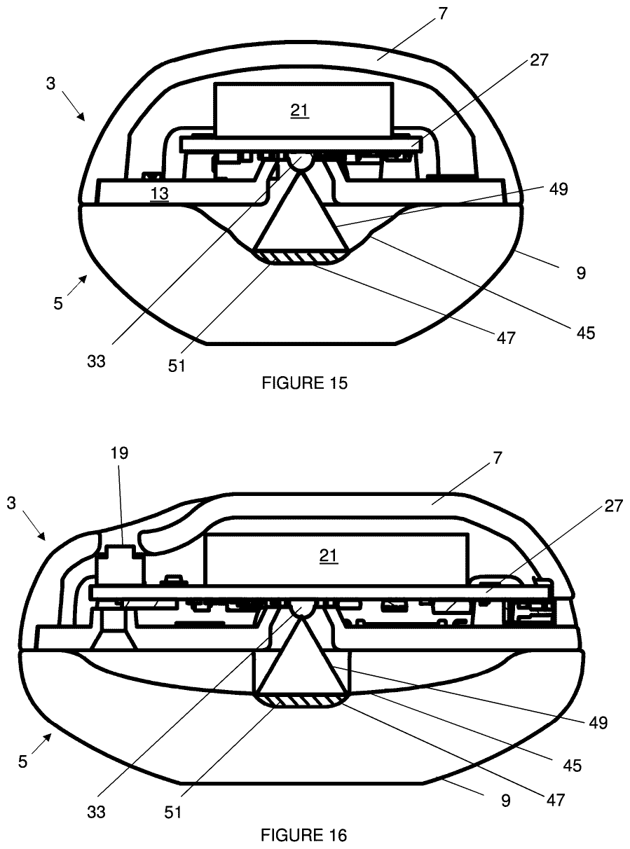

An example of the ultraviolet light emitting device 1 is provided in FIGS. 1 to 16 and comprises a housing including a first housing body 3 and a second housing body 5 that are capable of engagement with one another. As best shown in FIG. 2, the first body 3 includes a top cover 7, irradiation structure 11 and a base 13. The top cover 7 of the first housing body 3, best shown in FIGS. 3 and 4, includes a control button aperture or activator button aperture or control switch access opening 15 and a Universal Serial Bus (USB) port opening 19. The irradiation structure 11, best shown in FIGS. 2, and 5 to 10 includes a control button or control switch 17 to activate and deactivate the UVB device 1 thereby activating and deactivating irradiation, a power source 21, a USB port 23, and indicator light emitting diode(s) (LED(s)) 25 at an operatively top surface of a supporting body 27a and situated operatively between the supporting body 27 or a top surface of the supporting body 27a of the irradiation structure 11 and the top cover 7. In some embodiments of the invention, the irradiation structure 11 may include a printed circuit (PC) board as shown in FIG. 7 to 10, alternatively the supporting body 27 may be a PC board. Apertures 29, which may be PC board attachment apertures, are located on a periphery of the supporting body 27 to connect the irradiation structure 11 to connecting structures 31, which may be standoffs for PC board attachments, on the base 13. The control switch 17 may activate a timer which activates irradiation for a predetermined period of time. The indicator LEDs 25 may indicate to a user that the device is functioning or active and the battery level of the device or when a recharge or change or battery may be necessary.

The control switch may include a control mechanism which may be in communication with a cellular device application (app) or other remote device which controls activation of the ultraviolet light emitting device and irradiation remotely via remote communication methods such as Bluetooth or wireless networking technology. The cellular device application or other remote computing device may be used to control and monitor the device's performance.

The ultraviolet light emitting device may include measuring components or one or more sensors which measure battery levels, topical product levels, previtamin D or vitamin D delivery levels, or the like. These measuring components may be linked to the indicator LEDs and/or the application used to control and monitor the ultraviolet light emitting device. In another example embodiment red indicator LEDs may indicate the battery or power source as depleted and green indicator lights may indicate the battery or power source is fully charged. The application may then further include indicator components or screens or characters which display values measured by the measuring components such as the battery level of the ultraviolet light emitting device, low or too high levels of topical product within the recess, previtamin D or vitamin D delivery levels, or the like. In other words, the app executed at the computing device (e.g., on a mobile device) may also display battery level, alert a user to reorder the topical product, and/or let the user know how much Vitamin D has been delivered over a time frame.

At an operatively bottom surface of the supporting body 27b of the irradiation structure 11, the irradiation structure 11 includes an ultraviolet light source 33, such as a light emitting diode (LED), as is best shown in FIGS. 8, 9, 15 and 16. The ultraviolet light source may be capable of emitting ultraviolet B (UVB) light 49 having a wavelength in the range of 280 nanometres up to 315 nanometres, or 285 up to 310 nanometres. The ultraviolet light source 33 is located on or at the irradiation structure 11 such that the light source 33 and irradiation zone 47 are substantially opposite one another, as shown in FIGS. 8, 9, 15, and 16, so as to enable irradiation of the topical product 51 by the ultraviolet light source when the device is in an engaged condition whereby the first housing body 3 and the second housing body 5 are connected or engaged with one another.

The ultraviolet light source 33 may be a single LED or a plurality of LEDs, which may be located at a single point on the irradiation structure, for example in the middle of the operatively bottom surface of the support body 27b across from the irradiation zone 47 or dispersed across the bottom surface of the support body 27b.

In this embodiment, the power source 21 includes a rechargeable battery, which may be charged via the USB port 23 once connected to an external power source (not shown). The external power source may be a mains power source (not shown). In another embodiment (not shown) the power source may be a battery and exclude the USB charging port. In a further embodiment, the power source 21 may include a direct connection, either by means of a USB or other suitable port, to an external power source or mains power source. The USB port may be one of a USB-A, USB-B, USB-C, micro-USB, and mini-USB.

As shown in FIGS. 2, 5, 6, 11, and 12, the base 13 includes connecting means 31 for complementary connection or securing of the base 13 to the irradiation structure 11 via the apertures 29 on the supporting structure 27 of the irradiation structure 11. The base 13 further includes a safety device or safety mechanism 35a, 35b to prevent accidental irradiation and/or to alert a user that the device is active and irradiation is occurring, and magnet(s) 37 situated in apertures 39 of the base 13 to engage with magnet(s) 37 situated in apertures 41 of the second body 5. In alternative embodiments the magnet(s) 37 may be magnetic material.

The safety device may comprise a safety switch 35a, which may be a magnetic protection sensor, and/or safety light source 35b, wherein the ultraviolet light source 33 may be activated when the first 3 and second 5 housing bodies are engaged to activate the switch 35a, and deactivated when the first 3 and second 5 bodies are disengaged to deactivate the switch 35a. The switch 35a may comprise a hall effect sensor and semiconductor, or a magnetic switch. In this embodiment, as shown in FIG. 2, the safety switch 35a includes a magnetic switch situated in an aperture on the base 13 and a magnet situated in an aperture on the second housing body arranged to be located substantially opposite to one another when the device is in an engaged condition or closed configuration.

The base 13 of the first body 3 may further include a UV lens 43 within an aperture in the base 13 to enable ultraviolet light to travel from the ultraviolet light source 33 of the irradiation structure 11 though the UV lens 43 and irradiate a topical product located in the irradiation zone 47 of the second housing body 5. The UV lens may focus the ultraviolet light on the irradiation zone.

The magnet(s) 37 on the base 13 of the first body 3 and the second body 5 may form an engagement structure configured to releasably secure the first 3 and second 5 body. The magnet(s) on either of the first 3 or second 5 housing bodies may be arranged to releasably engage magnetic material in the other of the first and second housing bodies. Alternatively, or additionally, the one or more magnet(s) may be arranged to releasably engage a corresponding magnet in the other of the first and second housing bodies. The engagement structure may facilitate pivotal movement and/or sliding movement of the first and second housing between the open or disengaged condition and the closed or engaged condition.

In the engaged condition, the magnet(s) 37 of the first 3 and second 5 housing bodies are engaged or connected, preferably activating the switch of the safety device 35a and/or activating the safety device light source 35b to alert a user of activation of the ultraviolet light source 33. Upon engagement of the first and second housing bodies 3,5 the safety device 35a, 35b is also engaged whereby the switch 35a is activated when the device 1 is in the engaged condition which may in turn permit activation of the ultraviolet light and safety device light source 35b to alert a user that the device is active, and irradiation will occur. This warns a user of the potential danger faced should irradiation occur and prevents unwanted ultraviolet damage to a user].

In the disengaged condition, and in an exemplary version of the device 1 the magnet(s) 37 of the first 3 and second 5 housing bodies may be disengaged or disconnected, thereby deactivating the switch of the safety device 35a and/or deactivating the safety device light source 35b to alert a user of deactivation of the ultraviolet light source 33. This disengagement may be facilitated through pivotal movement or sliding movement of the first 3 and second 5 housing bodies relative to one another to disconnect the magnet(s) 37.

It is envisaged that another version of thee device for irradiating a topical product may be possible. In such a version, the second housing body may be a tray arrangement, wherein the second housing body is slidable into and out of the first housing body. In other words, the first housing body may be slidable between a disengaged condition where is it is fully located within the second housing body and an engaged condition where it is partially located within or removed from the second housing body. In this example arrangement, the recess may be located at or on the second housing body and filled with the topical product, after which the second housing body is slid into the first housing body to allow for irradiation of the topical product.

Referring again to FIGS. 1 to 12, upon disengagement of the first and second housing bodies 3,5 or substantial removal of the first housing body 3 from the second housing body 5 the safety device 35a, 35b is also disengaged whereby the switch 35a is deactivated which in turn deactivates the ultraviolet light source 33 and the safety light 35b to notify a user that the device is no longer active, and irradiation is now disabled. This may warn a user that the potential danger of irradiation is now eliminated or deactivated.

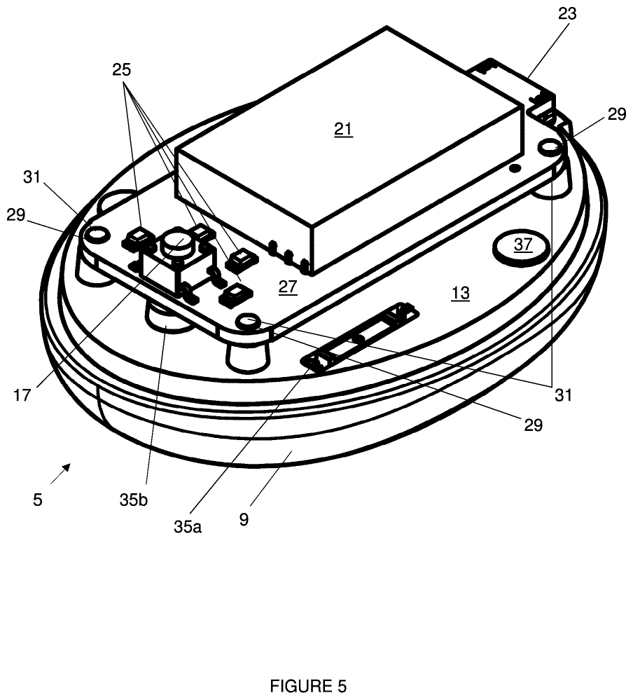

In the depicted version of e.g., FIG. 12, the second body 5 includes a bottom cover 9 (see FIG. 5), a recess 45 defined by the housing and configured to receive a topical or skincare product for irradiation. The recess 45 may include or define an irradiation zone 47. Upon engagement of the first 3 and second 5 housing bodies with one another in the closed or engaged condition, the ultraviolet light source 33 and irradiation zone 47, which may include the topical product, may be arranged such that the light source 33 and irradiation zone 47 are substantially opposite one another so as to enable irradiation of the topical product by the ultraviolet light source 33 to operatively convert a sterol or precursor 5,7-dehydrosterol in the topical product to an intermediate of vitamin D, for example previtamin D.

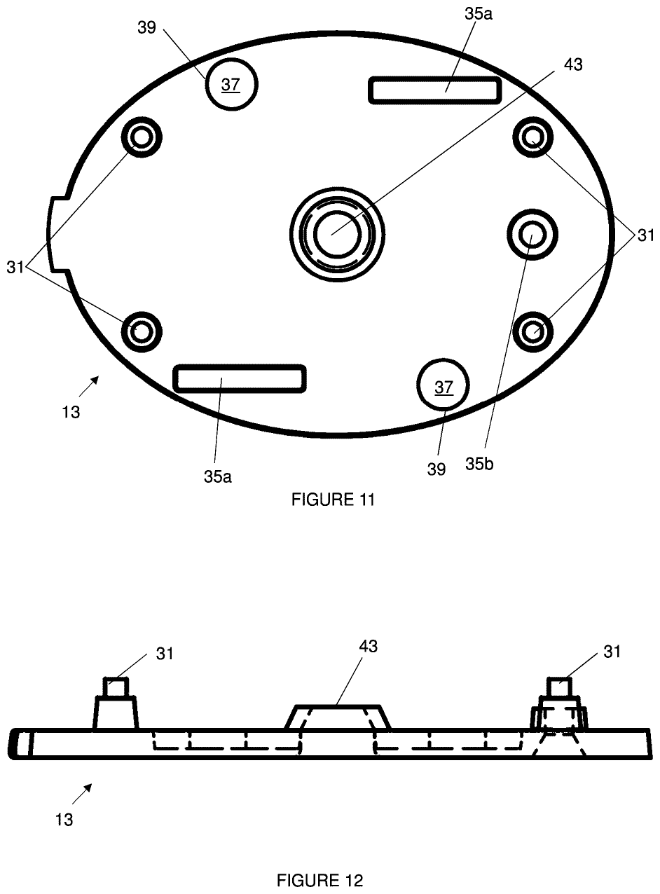

In use, the recess 45, located at the second housing body 5 in this embodiment, is filled with an amount of topical product 51, which may be in the form of a liquid, fluid, cream or serum. The first housing body 3 is then engaged with the second housing body 5 via the engagement structure, either by pivotal movement or sliding movement of the first housing body 3 relative to the second housing body 5, such that the magnet(s) 37 located in either body are engaged or connected and the ultraviolet light emitting device 1 is in the closed or engaged condition, as is best shown in FIGS. 11 and 12.

Once the recess 45 is filled with the amount of topical product 51, the topical product 51 runs to or flows into the irradiation zone 47 located at or within the recess 45 substantially opposite to light source 33, when the device 1 is in the engaged condition, so as to enable irradiation of the topical product 51 by the ultraviolet light source 33 to operatively convert a sterol or precursor 5,7-dehydrosterol in the topical product 51 to previtamin D. This filling of the irradiation zone 47 may occur before, during or after engagement of the first and second housing bodies 3,5 in the engaged condition once at least the second housing body 5 is placed on a substantially flat surface.

The depth of the recess 45 may vary such that the irradiation zone 47 is substantially deeper than the portion of the recess 45 adjacent to the irradiation zone 47 or surrounding the irradiation zone 47 to enable the topical product 51 to run towards or flow into the irradiation zone 47 and not to remain within the recess 45. The depth of the recess 45 may become gradually deeper toward the irradiation zone 47 to facilitate flow of the topical product 51. A step may be located between the irradiation zone 47 and the adjacent or surrounding recess 45.

Disengagement of the housing bodies 3, 5 may be complete disengagement, which is possible with the embodiment as shown in the exemplary embodiment of FIGS. 1 to 12. In a further embodiment disengagement may be only partial disengagement of the bodies 3, 5 wherein the engagement structure includes a pivot or slidable engagement (not shown) which retains a part of the housing bodies in the engaged condition i.e. in contact with one another, but wherein the bodies 3,5 disengage to enough of a degree that a topical product 51 may still be placed within the recess 45 and/or irradiation zone 45, after which the housing bodies 3, 5 may once again be placed via a pivotal or sliding movement into the engaged condition whereby the magnet(s) 37 and/or magnetic material on the bodies 3, 5 is connected.

In an alternative embodiment (not shown), the recess may be the irradiation zone, and the housing may include a plurality of ultraviolet light sources substantially opposite to the full area of the recess or irradiation zone such that topical product does not first need to flow to an irradiation zone located at or within the recess before irradiation of the topical product may occur.

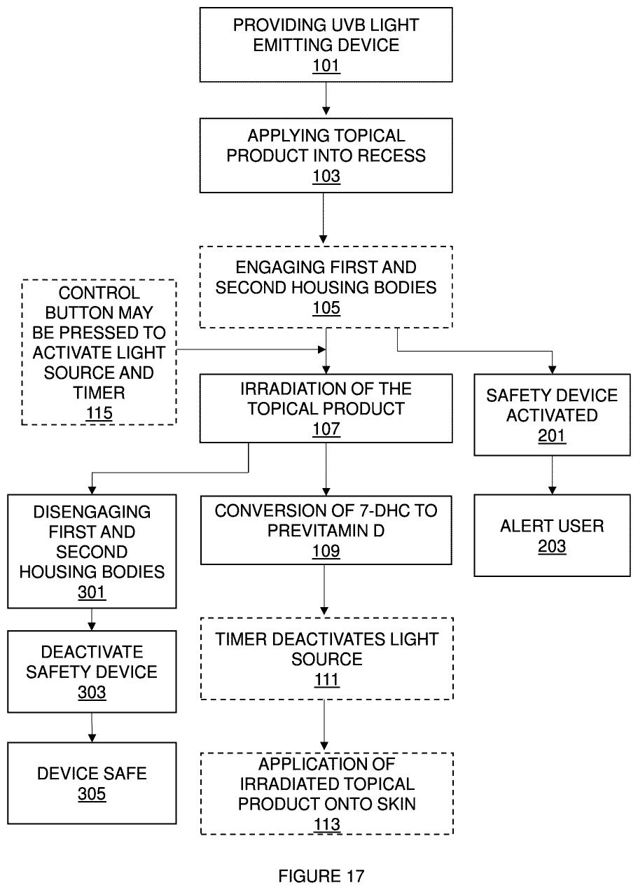

An example of a method for irradiating a topical product is provided in FIG. 17 which utilizes an exemplary ultraviolet light emitting device, e.g. that as shown in FIGS. 1 to 12. The method may include providing an ultraviolet light emitting device 101. An amount of the topical product may then be applied into the recess of the housing 103. Optionally, the first and second housing bodies may, optionally, be engaged with one another in the engaged condition 105 so as to cause irradiation of the topical product. Irradiation of the topical product may cause conversion of a sterol such as 7-Dehydrocholesterol (7-DHC) or provitamin-D or provitamin-D3 to photochemically convert to previtamin D or previtamin D3 or similar intermediate of vitamin D within the topical product while irradiated within the device. The previtamin D may then be converted to vitamin D by a user's body temperature, e.g., once administered to the skin.

In response to the engagement of the first and second housing bodies, the safety device may be triggered or activated 201 whereby the switch may be activated by engagement of the bodies which may, in turn, permit activation of an ultraviolet light source (not shown) and irradiation, and it may further activate the safety light source to alert a user 203 of irradiation. This activation of the safety device may optionally be caused by the engagement of a protection circuit (not shown).

The irradiation may optionally be activated by a user pressing the control button or switch 115 which in turn activates the ultraviolet light source and/or a timer to then permit irradiation of the topical product 107. The timer may activate irradiation for a predetermined period of time. Once UVB conversion of the 7-DHC to previtamin D is complete, the timer deactivates 111 which may in turn deactivate the light source. Upon this deactivation of the light source a user may optionally receive an alert. Following the alert, a user may remove the irradiated topical product from the device and apply the product to their skin 113.

Once irradiation is completed and conversion of the sterol to previtamin D or similar intermediate of vitamin D completed, the first and second bodies may once again be disengaged 301, after which the irradiated topical product may be removed. The disengagement of the housing bodies may deactivate the safety device 303 as the switch is turned off and in response the ultraviolet light source or irradiation and/or also the safety light source may be disabled or deactivated. The device may then be rendered safe 305 in the disengaged condition thus accidental irradiation may be prevented.

The step of engaging the first and second housing bodies 105 may be facilitated by pivotal movement and/or sliding movement of an engagement structure between the open or disengaged condition and the closed or engaged condition.

The conversion rate of 7-dehydrocholesterol in a fatty acid ester solvent to previtamin D or similar intermediate of Vitamin D may occur at a rate of about 10% to 99% in 2 seconds to 24 hours, or it may occur substantially in 10 seconds up to 10 minutes, or in 1 second up to 10 hours.

An example of a topical product for use with the device and method is provided. The topical DHC product comprises 7-dehydrocholesterol dissolved in a skin-compatible solvent. The skin-compatible solvent may have low ultraviolet absorption in the range 285 to 310 nanometres, and may comprise a fatty acid ester, an alcohol, or both of an ester of isoamyl alcohol and a fatty acid.

The fatty acid included in the topical product may be lauric acid. In a further embodiment, the skin-compatible solvent may be one of isoamyl laurate, isopropyl palmitate, isopropyl myristate, octyl dodecanol, C12-C15 alkyl benzoate, or combinations thereof.

The advantages provided by the exemplary ultraviolet device and method include that the device may decrease the need for exposure to direct sunlight, and it may allow for vitamin D synthesis in humans without the harmful effects of ultraviolet light exposure or the risks of supplementation with synthetic vitamin D. A further advantage may be the inclusion of a safety device or switch which prevents the activation of the ultraviolet light source and thus irradiation unless the device is closed or engaged condition thus containing the ultraviolet irradiation and preventing accidental irradiation. This way, the device can be prevented from functioning unless it is in the closed or engaged condition. In other words, the safety device may act to deactivate the device when the device is in the open or disengaged condition.

By applying the irradiated topical product, which includes previtamin D, onto a user's skin an improvement of certain skin conditions associated with low vitamin D levels may be observed as well as an overall increase in systemic vitamin D levels. The device may therefore obviate excessive exposure to UV light (sunlight or artificial light) and the associated risks of an increased rate of ageing, DNA damage, and melanoma or skin cancer.

The foregoing description has been presented for the purpose of illustration; it is not intended to be exhaustive or to limit the technology to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the present disclosure be limited not by this detailed description, but rather by any claims that issue on an application based hereon. Accordingly, the present disclosure is intended to be illustrative, but not limiting, of the scope of any accompanying claims.

Finally, throughout the specification and any accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. An ultraviolet light emitting device for irradiating a topical product, the device comprising:
a housing;
a recess defined by the housing and configured to receive the topical product; and
an ultraviolet light source capable of irradiating the topical product within an irradiation zone of the recess,
wherein the light source and irradiation zone are substantially opposite one another so as to enable irradiation of the topical product by the ultraviolet light source to operatively convert a sterol in the topical product to an intermediate of vitamin D.

2. The ultraviolet light emitting device as claimed in claim 1, wherein the housing comprises at least a first housing body and a second housing body.

3. The ultraviolet light emitting device as claimed in claim 1, wherein the sterol is 5,7-dehydrosterol.

4. The ultraviolet light emitting device as claimed in claim 1, wherein the sterol is 7-dehydrocholesterol.

5. The ultraviolet light emitting device as claimed in claim 1, wherein the ultraviolet light source is capable of emitting ultraviolet B (UVB) light.

6. The ultraviolet light emitting device as claimed in claim 5, wherein the UVB light has a wavelength in the range of 280 nanometres up to 315 nanometres.

7. The ultraviolet light emitting device as claimed in claim 1, wherein the ultraviolet light source is a light emitting diode (LED) light.

8. The ultraviolet light emitting device as claimed in claim 1, wherein the intermediate of vitamin D is an intermediate isomer or previtamin D or previtamin D₃ or tachysterol or lumisterol.

9. The ultraviolet light emitting device as claimed in claim 2, wherein the light source is located at the first housing body, and the irradiation zone at the second housing body.

10. The ultraviolet light emitting device as claimed in claim 9, wherein the light source and irradiation zone are both located at one of the first and second housing bodies.

11. The ultraviolet light emitting device as claimed in claim 2, wherein the housing includes an engagement structure configured to releasably secure the first and second housing bodies.

12. The ultraviolet light emitting device as claimed in claim 11, wherein the engagement structure is in the form of one or more magnet(s) or magnetic material provided in either of the first and second housing bodies.

13. The ultraviolet light emitting device as claimed in claim 12, wherein the one or more magnet(s) or magnetic material are arranged to releasably engage magnetic material in the other of the first and second housing bodies, and/or wherein the one or more magnet(s) are arranged to releasably engage a corresponding magnet in the other of the first and second housing bodies.

14. The ultraviolet light emitting device as claimed in claim 12, wherein the engagement structure is an array of magnets or magnetic material disposed along a periphery of an internal surface of either of the bodies and configured to magnetically secure the bodies to one another.

15. The ultraviolet light emitting device as claimed in claim 1, wherein the device includes a safety device to prevent accidental irradiation.

16. The ultraviolet light emitting device as claimed in claim 15, wherein the safety device comprises a magnetically activated switch configured to deactivate the ultraviolet light source when the first and second bodies are disengaged.

17. The ultraviolet light emitting device as claimed in claim 15, wherein the switch comprises a hall effect sensor and semiconductor or a reed switch.

18. The ultraviolet light emitting device as claimed in claim 1, wherein the device includes a power source.

19. The ultraviolet light emitting device as claimed in claim 18, wherein the power source is one or more of a battery, a rechargeable battery, a Universal Serial Bus (USB) port for connection to an external power source, and a mains power source.

20. The ultraviolet light emitting device as claimed in claim 4, wherein the conversion rate of 7-dehydrocholesterol in a fatty acid ester solvent to an intermediate of vitamin D occurs at a rate of about 10% to 99% in about 2 seconds up to 24 hours.

21. A method for irradiating a topical product, the method including the steps of
(i) providing an ultraviolet light emitting device as claimed in claim 1;
(ii) applying topical product into the recess of the housing; and
(iii) enabling irradiation of the topical product by the ultraviolet light source so as to operatively convert a sterol in the topical product to an intermediate of vitamin D.

22. A topical product for irradiation for use with the device as claimed in claim 1, comprising 7-dehydrocholesterol dissolved in a skin-compatible solvent.

23. The topical product for irradiation as claimed in claim 22, wherein the skin-compatible solvent has low ultraviolet absorption in the range 280 to 315 nanometres.

24. The topical product for irradiation as claimed in claim 22, wherein the skin-compatible solvent is a fatty acid ester.

25. The topical product for irradiation as claimed in claim 22, wherein the skin-compatible solvent is an alcohol.

26. The topical product for irradiation as claimed in claim 22, wherein the skin-compatible solvent is an ester of isoamyl alcohol and a fatty acid.

27. The topical product for irradiation as claimed in claim 22, wherein the fatty acid is lauric acid.

28. The topical product as claimed in claim 27, wherein the solvent is one of isoamyl laurate, isopropyl palmitate, isopropyl myristate, octyl dodecanol, C12-C15 alkyl benzoate, or combinations thereof.
